(19) United States
(12) Patent Application Publication
(10) Pub. No.: US 2002/0159984 A1
(43) Pub. Date: Oct. 31, 2002

(54) Cultivation of cells for long term engraftment

(75) Inventor: Ronald Brown, Derwood, MD (US)

Correspondence Address: BIRCH STEWART KOLASCH & BIRCH, PO BOX 747, FALLS CHURCH, VA, 22040-0747, US

(73) Assignee: Quality Biological, Inc.

(21) Appl. No.: 10/134,334

(22) Filed: Apr. 30, 2002

Related U.S. Application Data
(62) Division of application No. 09/438,964, filed on Nov. 12, 1999.

(51) Int. Cl. G01N 33/567; A61K 48/00

(52) U.S. Cl. 424/93.21; 435/7.21

(57) ABSTRACT

In the art of tissue culture, it has been desired that serum-free culture conditions be found that support the growth and proliferation of hematopoeitic stem cells ex vivo. The present invention discloses a serum-free medium comprised, for example, of pharmaceutical grade components including pasteurized human proteins, that in the presence of the appropriate growth factors, supports the ex vivo maintenance, proliferation and/or differentiation of CD34⁺/CD38⁻ cells derived from cord blood, mobilized peripheral blood or bone marrow. In conjunction with this effort, the ability of serum-free medium to maintain or cause proliferation of the HSCs ex vivo is assessed by their long-term engraftment in a chimeric sheep animal model.

This application is a divisional of co-pending application Ser. No. 09/438,964, filed on Nov. 12, 1999, the entire contents of which are hereby incorporated by reference.

This invention was made with Government support under Small Business Innovative Research Grant No. R44CA76832 awarded by the National Cancer Institute of the National Institute of Health. The government has certain rights in the invention.

### BACKGROUND OF THE INVENTION

Every blood cell originates from a single cell type in the bone marrow of adult animals during a process known as haemapoiesis. These cell types are called hematopoietic stem cells (HSCs) and are normally found in normal human bone marrow (BM), the peripheral blood (PB) of patients treated with cytokines (termed mobilized CD34⁺ cells), and umbilical cord blood (CB). As multipotent cells, HSCs give rise to specialized cell types but renew themselves by cell division such that their population remains constant. After development in the bone marrow, the differentiated cells are transported throughout the body by the bloodstream.

In the art of tissue culture it has been desired that serum-free culture conditions be found that support the growth and proliferation of HSCs. In fact, many therapeutic regimes are being developed which depend upon the maintenance and growth of HSCs ex vivo, as the transplantation of these cells is a highly effective treatment for several human diseases, including hematologic malignancies, marrow failure syndromes, congenital immunodeficiencies, and some metabolic disorders.

Histocompatible related allogeneic marrow transplantation has been a successful therapy for patients with hematologic malignancies, aplastic anema, severe congenital immunodeficiency states, and selected inborn errors of metabolism (Leary 1987; Rowley 1987). Furthermore, some cancer therapies, such as high-dose chemotherapy or radiation, deplete HSCs and may necessitate a bone marrow transplant in order to replenish the HSC population. In the case of an autologous BM transplant, the BM must be removed from the patient and transplanted back into the patient. Alternatively, the BM may be frozen prior to therapy, and then thawed before transplantation. Significant risks associated with this procedure are the possibility that the BM may contain tumor cells, or that the BM does not contain an adequate amount of cells to sufficiently repopulate the patient.

While autologous marrow has been the traditional source of stem cells, the use of matched, related allogeneic HSCs has also been used as the source of transplantable stem cells (Iscove, 1989; Smith 1991; Flake 1986; Zanjani 1992). However, the major limitation using HLA-matched sibling donors is that only about 30% of patients have a matched donor. Furthermore, the number of donors is limited because of HLA polymorphism and ethnic diversity. These limitations have necessitated that new methods to obtain HSCs be investigated. From this, umbilical cord blood arose as a promising source of stem cells for transplantation.

The use of umbilical cord blood as an alternative source of HSCs was first reported in 1989 (Gluckman 1989). Since then, more than 500 such transplants have been performed in the United States and Europe. The advantages of umbilical cord blood are that CB is enriched in primitive HSCs, which facilitates engraftment, and that CB cells are immunologically immature, which decreases the likelihood and severity of graft-vs.-host disease. Furthermore, umbilical cord blood HSCs have distinct proliferative advantages, including increased cell cycle rate, the production of autocrine growth factors by the HSCs, and an increased telomere length. Furthermore, the small number and relative immaturity of cord blood T-cells may reduce the risk of graft vs. host disease, permitting a relatively high degree of HLA disparity between the donor and recipient.

A serious and common disadvantage of using umbilical cord blood as the source of HSCs is that it contains a reduced number of stem cells compared to bone marrow as determined by enumeration of nucleated mononuclear cells and/or CD34⁺ cells. Too few stem cells may not allow for engraftment in adult patients, which is evidenced by some reports that suggest ex vivo expansion of umbilical cord blood progenitors may be necessary for larger patients (Wingo 1995). In vitro studies have shown that ex vivo expansion of HSCs is possible. In addition, umbilical cord blood progenitor cells may be more sensitive to expansion with both lineage-specific and lineage-nonspecific hematopoietic growth factors (Zanjani 1996; Fisher 1985; Fisher 1989; Veronesi 1981; Valagussa 1978 Fisher 1984). Furthermore, CD34⁺ stem cells can be isolated and expanded from umbilical cord blood while retaining a level of self-renewal capacity (Fisher 1984; Nemoto 1980; Hortobagyi 1992).

HSCs that are known in the art to be suitable for long-term engraftment are CD34⁺/CD38⁻ stem cells (Zanjani 1996). It has been shown that a subpopulation of progenitor cells found in umbilical cord blood having the CD34⁺/CD38⁻ phenotype has a higher cloning efficiency and proliferates in culture more rapidly than those CD34⁺/CD38⁻ cells of the adult bone marrow (Hao et al. 1995). However, these cells were never utilized for long-term engraftment. Furthermore, there are two main obstacles to using these cells for transplantation purposes. The first is that these cells are not found in sufficient amounts in cord blood or bone marrow to allow for adult transplants. The second is that when these cells were cultured in a serum-containing medium, they differentiate into CD34⁺/CD38⁺ cells, which are not acceptable for long-term engrafting purposes.

### SUMMARY OF THE INVENTION

In view of the above considerations, a need exists in the art to develop culture conditions that allow for the cultivation of CD34⁺/CD38⁻ ex vivo for long-term engraftment and other purposes. Thus, in one aspect, the present invention relates to a method to support the growth and proliferation of normal human HSCs ex vivo that are suitable for long-term engraftment.

The development of a well-defined culture medium to cultivate the HSCs and a reliable in vivo testing system to determine their long-term survival is critical to this process. The present invention discloses a serum-free medium comprised, for example, of pharmaceutical grade components including pasteurized human proteins, that in the presence of the appropriate growth factors, supports the ex vivo maintenance, proliferation and/or differentiation of CD34⁺/CD38⁻ cells derived from cord blood, mobilized peripheral blood or bone marrow. In conjunction with this effort, the ability of serum-free medium to maintain or cause proliferation of the HSCs ex vivo is assessed by their long-term engraftment in a chimeric sheep animal model.

It is therefore one object of the invention to provide a method for preparing a population of cells maintained or enriched in CD34⁺ mammalian cells and suitable for long-term engraftment, comprising the step of culturing mammalian hematopoietic stem cells in a serum-free medium comprising a basal media, an effective amount of one or more cytokines and an effective amount other essential nutrients to maintain or enrich said population of cells in CD34⁺ cells. Preferably, the CD38⁻ cells will proliferate without expressing the CD38⁺ phenotype, which is indicative of cell differentiation. Also, the CD34⁺/CD38⁻ cells will preferably not lose their long-term engrafting capability.

It is another object of the invention to provide a method for repopulating the hematopoietic stem cell population of a patient comprising transplanting cells cultured according to the method as described above into said patient. This cell population used for transplant may be prepared by culturing a cell sample comprising hematopoietic stem cells for at least two days under serum-free conditions.

This invention also provides a method for determining the suitability of a cell population for transplantation into a patient comprising the steps of obtaining a sample of cells including hematopoietic stem cells and determining the number of CD34⁺/CD38⁻ cells in said sample.

Furthermore, this invention also describes a method of gene therapy, comprising the steps of culturing a sample of hematopoietic stem cells under conditions which maintain an effective amount of cells having the long-term engraftment phenotype, transferring a therapeutic gene to correct a genetic defect into said hematopoietic stem cells either before or after said culturing, and then transplanting a therapeutic amount of hematopoietic stem cells into a patient requiring said gene therapy.

It is another object of the invention to provide a method of transporting hematopoietic stem cells without cryopreservation, comprising the step of placing said cells in a serum-free medium comprising an effective amount of at least one cytokine and an effective amount of a basal media containing other essential nutrients for growth or maintenance, and transporting said cells at a temperature between 4° C. and 40° C.

It is another object of the invention to provide a kit for expanding a population of hematopoietic stem cells comprising the components of serum-free medium instructions for culturing HSCs under conditions that expand the number of hematopoietic stem cells.

### BRIEF DESCRIPTION OF THE DRAWINGS

FIGS. 1A and 1B. Kinetics of expansion of CD34⁺ cells. CD34⁺ cells were cultured for 3, 7 or 14 days in QBSF-60 with or without serum, in the presence of IL-3, IL-6 and SCF.

FIG. 1A shows the amount of proliferation of a human adult BM-enriched CD34⁺ population at various time points in both serum-free and serum-containing medium.

FIG. 1B shows the fold increase in total cell numbers cultured in both serum-containing and serum-free medium. Cell counts were taken at 3, 7 and 14 day time points of incubation.

FIG. 2. Fold increase in CD34⁺ cells in culture. FIG. 2 shows the fold increases in the amount of CD34⁺ cells in both serum-containing and serum-free medium. Cell counts were taken at day 3, 7 and 14 of incubation.

FIG. 3. Fold increase in CD34⁺/CD38 cell counts in culture. FIG. 3 shows the fold increase of CD34⁺/CD38⁻ cell counts over 14 days of culture. Cells were cultured in either serum-containing or serum-free medium, and cell counts were taken at day 3, 7 and 14 of incubation.

FIGS. 4A and 4B. Percentage of human cells in primary sheep transplant recipients.

FIG. 4A shows the percentage of human cells in the BM of the primary sheep transplant recipients sixty days post-transplant.

FIG. 4B shows the percentage of human cells in the PB of the primary sheep transplant recipients sixty days post-transplant. For both figures, cells were cultured for either 3, 7, or 14 days before transplant. All cells counts were taken at 60 days post-transplant.

FIG. 5. Percentage of human cells in the BM of primary sheep transplant recipients recorded over a time frame of 8 months. FIG. 5 shows the percentage of human cells in the BM of the primary sheep transplant recipients. Prior to transplant, cells were cultured for various time periods (0 days, 3 days, 7 days or 14 days), either in serum-containing or serum-free media (with or without). Cells counts were taken at various time points (60 days, 1 week, 3 months, and 8 months) post-transplant.

FIG. 6. Percentage of human cells in the BM of secondary sheep transplant recipients recorded over a time frame of 8 months. FIG. 6 shows the percentage of human cells in the BM of the secondary sheep transplant recipients. Prior to transplant, cells were cultured for various time periods (0 days, 3 days, 7 days or 14 days), either in serum-containing or serum-free media (with or without). Cells counts were taken at various time points (60 days, 3 months, and 8 months) post-transplant.

### DETAILED DESCRIPTION OF THE INVENTION

One aspect of the invention is a method to prepare a population of cells enriched in CD34⁺/CD38⁻ phenotypes for the purpose of long-term engraftment. Long term engrafting cells are cells which produce differentiated cells which are present in the peripheral blood of a transplant recipient while also maintaining a population of HSCs in the bone marrow of the recipient for 60 days. Long-term engrafting cells are usually detectable in a secondary graft recipient using the experimental conditions reported in this application. Several methods exist in which to measure long-term engraftment. In one method, human CD34⁺/CD38⁻ HSCs transplanted into the BM of a secondary transplant recipient sheep that persists at least 60 days post transplantation are considered long-term engrafting. Thus far, chimeric sheep transplanted with such cells have survived over 3.5 years. Alternatively, a selectable marker may be inserted into the HSCs that are used for stem cell transplant. If the marker persists in the subject receiving the transplanted HSCs containing the selectable marker for at least 60 days, then the cell containing that marker in the bone marrow is considered long-term engrafting.

As used herein the terms “enriched” or ‘enrichment’, when used in conjunction with the number of CD34⁺/CD38⁻ cells in a cell population, means that the total number of CD34⁺/CD38⁻ cells is constant or increasing in proportion to the total number of cells in the cell population. For therapeutic purposes, it is desirable to culture the cells under conditions which at least maintain, and preferably increase, the total number of such CD34⁺/CD38⁻ cells in culture. In accordance with the present invention, the total number of CD34⁺/CD38⁻ cells can be expanded between 3- and 14-fold with respect to the initial amount of cells.

The term “serum-free” is used herein to mean that all whole serum is excluded from the medium. Certain purified serum components, such as human serum albumin, can be added to the medium. For the purposes of this description, the term “effective amount” of growth factors and other components is that which allows for the maintenance or proliferation of CD34⁺/CD38⁻ hematopoietic stem cells in a serum-free culture, preferably for over three days in culture.

Hematopoietic Stem Cells Useful in the Invention

Umbilical cord blood is obtained from intact placentas following normal delivery. The umbilical cord blood may be obtained following delivery of the placenta or in utero following delivery of the infant. Ex utero collection consists of clamping the placenta and placing it along with the umbilical cord into a sterile pan. The umbilical cord is aseptically punctured and the umbilical cord blood drains by gravity into a collection bag. The collection bag contains CPD-A, which is a commercially available anticoagulant. After about 100 mls of blood is collected, the needle is removed from the umbilical cord and the tubing clamped. The placenta and cord can then be discarded. The cord blood cells may be used fresh or frozen for later use. Clinical studies typically utilize frozen blood. Cryopreservation of the umbilical cord blood is performed according to established procedures, using 10% final concentration of DMSO.

Bone marrow may be obtained by aspiration from most preferably the posterior iliac crest. Progenitor cells may also isolated from a donor or patient by treatment with filgrastim (granulocyte colony-stimulating factor, or G-CSF [Neupogen, Amgen, Munich, Germany]) at a dose of 5 μg per kilogram of body weight subcutaneously, which will mobilize peripheral-blood progenitor cells (Brugger 1993). The cells may be collected in a leukapheresis as described in Brugger (1993).

The starting population of HSCs used for ex vivo growth may be mixed, or selected by flow cytometry using a CD34⁺ cell-surface marker (FACScan analyzer, Becton Dickinson, Heidelberg, Germany). Alternatively, a population of CD34⁺-enriched hematopoietic cells may be bought frozen from a commercial source, such as Bio Whittaker (MD). Any source of human CD34⁺/CD38⁻ cells can be used in accordance with the present invention. Alternatively, a population of CD34⁺-enriched hematopoietic cells may be obtained via an immunoaffinity column affixed with a CD34⁺ monoclonal antibody (Smith et al. WO 95/06112). Whole blood typically contains about 1-2% of CD34⁺ cells, which may be separated from the whole blood by conventional techniques to prepare a cell population having a purity of 90% or more CD34⁺ cells. Of the commercially available CD34⁺ enriched cell populations, 90% or more of the cells are CD34⁺, of which typically 1-5% are phenotypically CD34⁺/CD38⁻.

Expansion of CD34⁺/CD38⁻ Cells Ex Vivo

The expansion of CD34⁺/CD38⁻ cells freshly obtained or from cryopreserved sources ex vivo as described herein preferably involves the use of clinical grade serum-free reagents and employs culture conditions (growth factor types, concentrations, length and temperature of culture). These parameters are being studied to optimize a system which allows for long-term engraftment of the HSCs.

Serum Free Medium

“Serum Free Medium” used herein comprises a Basal medium and other components necessary for maintenance and/or growth. Other such components are described below. A preferred media is QBSF-60, which is commercially available from Quality Biological, Inc., Gaithersburg, Md., and which is described in more detail below. The cytokines discussed herein are added to the QBSF-60 at appropriate concentrations. The composition of QBSF-60 is also described in detail in copending U.S. patent application Ser. No. 08/953,434, filed on Oct. 17, 1997, the entire contents of which are hereby incorporated by reference. QBSF-60 was the “serum-free” media used in the experiments reported in this application. However, other commercially available media may be used provided that the appropriate additives (including cytokines) are added to or are present in the media.

Basal Medium

The basal medium is preferably Iscove's modified Dulbecco's medium (IMDM). Other such basal media might be used, such as McCoy's 5a or a blend of Dulbecco's modified Eagle's Medium and Ham's-F12 media at a 1:1 ratio. The requirements of the basal medium are that it provide i) inorganic salts so as to maintain cell osmolality and mineral requirements (e.g., potassium, calcium, phosphate, etc.), ii) essential amino acids required for cell growth, that is, amino acids not made by endogenous cellular metabolism, iii) a carbon source which can be utilized for cellular energy metabolism, typically glucose, and iv) various vitamins and co-factors, such as riboflavin, nicotinamide, folic acid, choline, biotin, and the like, as my be required to sustain cell growth. Glutamine is one of the amino acids that may be added to the medium of the present invention in an effective amount. The glutamine concentration is usually between 100 and 500 μg/ml, preferably between 125 and 375 μg/ml and most preferably between 150 and 300 μg/ml. Because of its instability, glutamine is sometimes added just before use of the media.

The basal medium also typically contains a buffer to maintain the pH of the medium against the acidifying effects of cellular metabolism, usually bicarbonate or HEPES. The pH of the basal medium is usually between 6.8 and 7.2. The composition of IMDM is shown in Table I, below:

TABLE I
Iscove's Modified Dulbecco's Medium

| Component | mg/L |
|---|---|
| L-Alanine | 25.0 |
| L-Arginine HCl | 84.0 |
| L-Asparagine · H₂O | 28.40 |
| L-Aspartic Acid | 30.0 |
| L-Cystine · 2HCl | 91.24 |
| L-Glutamic Acid | 75.0 |
| L-Glutamine | 584.0 |
| Glycine | 30.0 |
| L-Histidine HCl.H₂O | 42.0 |
| L-Isoleucine | 104.8 |
| L-Leucine | 104.8 |
| L-Lycine HCl | 146.2 |
| l-Methionine | 30.0 |
| L-Phenylalanine | 66.0 |
| L-Proline | 40.0 |
| L-Serine | 42.0 |
| L-Threonine | 95.2 |
| L-Tryptophan | 16.0 |
| L-Tyrosine, 2Na.2H₂O | 103.79 |
| L-Valine | 93.6 |
| Biotin | 0.013 |
| D-Ca Pantothenate | 4.00 |
| Choline Chloride | 4.00 |
| Folic Acid | 4.00 |
| i-Inositol | 7.00 |
| Nicotinamide | 4.00 |
| Pyridoxal HCl | 4.00 |
| Riboflavin | 0.40 |
| Thiamine HCl | 4.00 |
| Vitamin B₁₂ | 0.013 |
| Antibiotics | Omitted |
| 2-a-Thioglycerol (7.5 E-5 M) | Omitted |
| CaCl₂.2H₂O | 215.86 |
| KCl | 330.0 |
| KNO₃ | 0.076 |
| MgSO₄ (anhyd) | 97.67 |
| NaCl | 4505. |
| NaH₂PO4 | 108.69 |
| Na₂SeO₃.5H₂O | 0.0173 |
| Glucose | 4500. |
| Phenol Red · Na | 15.34 |
| Sodium Pyruvate | 110.0 |
| NaHCO₃ | 3024. |
| HEPES 25 mM | 5958. |
| CO₂ (The air in the jar over | 5% |
| the medium contains 5% CO₂ | |
| and air) | |

Preparation of Media

The medium of the present invention is of course aqueous and is made using distilled water. The medium is formulated from freely soluble materials. Thus, the order of the addition of the ingredients is not particularly important to the invention. Typically, the basal medium is made first and the remaining components required for growth of bone marrow cells in the absence of serum are then added to the basal medium.

The most ideal system, as described in this invention, is one wherein the serum-free media is made fresh on the day that it is to be added to the culture. However, when storage previous to use is necessary, it may be desirable to add certain compounds. Reducing agents such as a-monothioglycerol and p-mercaptoethanol, which are thought to diminish free-radical formation, may be added to the serum-free media formulations. This will enhance stability of the serum-free media, allowing it to be stored for up to 20 days or longer lengths of time. Additionally, in these less than preferred circumstances, antibiotics may also be added to the media as a precaution against bacterial contamination.

All of the ingredients in the medium, including the ingredients in the basal medium, are present in amounts sufficient to support the maintenance, proliferation and/or differentiation of CD34⁺ cells, depending on the desired use. If a basal medium is made which comprises IMDM reformulated with respect to the amounts of the components of IMDM, it is expected that the reformulation will contain those essential components of IMDM in amounts 0.1 to 10, preferably 0.5 to 2 times, most preferably 0.8 to 1.2 times their amounts in the formulation IMDM described above.

In order to develop a medium that can be used for human clinical CD34⁺ cell regimens, the components of media developed for unfractionated bone marrow should be optimized with U.S. Pharmaceutical grade components. The serum-free media of this application (QBSF-60) is composed of the basal medium IMDM plus the following additives, 2 mM L-glutamine, 100 U/ml penicillin, 100 μg/ml streptomycin and the serum-free components: human injectable grade serum albumin (4 mg/ml) (Alpha Therapeutic Corporation), partially iron saturated human transferrin (300 μg/ml) (Serologicals, Inc.) and human recombinant sodium insulin (0.48 U/ml) (Sigma). Since L-glutamine present in IMDM is unstable, additional glutamine was added to the medium.

The medium is formulated and sterilized in a manner conventional in the art. Typically, stock solutions of these components are made filter sterilized. A finished medium is usually tested for various undesired contaminants, such as mycoplasma or virus contamination, prior to use.

Growth Factors

The presence of appropriate growth factors in the medium, such as interleukins (IL), colony stimulating factors (CSF), and the like, will influence the rate of proliferation and the distribution of cell types in the population. Cytokines used for the expansion and differentiation of early progenitor cells are FLT-3 ligand stem cell factor, thrombopoietin (TPO), interleukin-1 (IL-I) and interleukin-6 (IL-6). Cytokines used to stimulate proliferation and differentiation of mid-progenitor cells are interleukin-3, granulocyte colony-stimulating factor (G-CSF), and granulocyte-macrophage colony stimulating factor (GM-CSF). Cytokines that promote the differentiation of specific blood cell types are G-CSF, macrophage colony stimulating factor (M-CSF) and erythropoietin. Development of a myeloid population, especially GM-colony forming cells, is highly desirable for the transplant patient to survive since these cells are responsible for fighting infections.

The role which each of these cytokines play in hematopoiesis is under intense investigation in the art and it is expected that eventually it will be possible to faithfully recapitulate hematopoiesis ex vivo. Various growth factors and/or cytokines for driving proliferation of the cells can be added to the medium used to culture the cells. By means of adding various cytokines at different stages of the culture, the cell population can be altered with respect to the types of cells present in the population by following the teachings of U.S. Pat. No. 5,846,529 (Nexell), the entire contents of which are hereby incorporated by reference. Cytokines should be selected to promote the maintenance and/or expansion of CD34⁺/CD38⁻ cells. In order to accomplish this, one or more of the following cytokines can be added to the media: FLT3, STF, IL-I, IL-6, TPO, etc.

Cytokines used in the present invention were present in effective amounts, usually ranging from 0.1 to 200 ng/ml, preferably 1-100 ng/ml, and most preferably 1-50 ng/ml. The amount of IL-3 added to the medium usually ranges from 0.1 to 100 ng/ml, is preferably 1-20 ng/ml, and is most preferably 5 ng/ml. The amount of SCF (used in combination with IL-3 and IL-6) added usually ranges from 0.1 to 100 ng/ml, is preferably 1-50 ng/ml, and is most preferably 10 ng/ml. The amount of IL-6 added to the medium usually ranges from 0.1 to 100 ng/ml, is preferably 1-20 ng/ml, and is most preferably 5 ng/ml.

The amount of TPO added to the medium usually ranges from 0.1 to 200 ng/ml, is preferably 1 to 100 ng/ml, and is most preferably 10 ng/ml. The amount of FLT3 added is preferably in the range of 0.1 to 200 ng/ml, is preferably 1 to 100 ng/ml, and is most preferably 10 ng/ml. The amount of SCF (used in combination with TPO and FLT3) added ranges from 0.1 to 100 ng/ml, is preferably 1 to 50 ng/ml, and is most preferably 5 ng/ml.

Albumin/Source of Nutrients

Albumin is preferably supplied in the form of human serum albumin (HSA) in an effective amount for the growth of cells. HSA provides a source of protein in the media. Moreover, protein acts as a substrate for proteases that might otherwise digest cell membrane proteins. Albumin is thought to act as a carrier for trace elements, essential fatty acids, and cholesterol. HSA is greatly advantageous over protein derived from animals such as bovine serum albumin (BSA) due to the reduced immunogenic potential of HSA. The HSA may be derived from pooled human plasma fractions, or may be recombinantly produced in such hosts as bacteria and yeast, or in vegetable cells such as potato and tomato. Preferably, the HSA used in the present formulations is free of pyrogens and viruses, and is approved regulatory agencies for infusion into human patients. The HSA may be deionized using resin beads prior to use. The concentration of human serum albumin is usually 1-8 mg/ml, preferably 3-5 mg/ml, most preferably 4 mg/ml. However, the exact amount of albumin may vary depending upon the type of albumin used.

Soluble Carrier/Fatty Acid Complex

The albumin mentioned above could be substituted by a soluble carrier/essential fatty acid complex and a soluble carrier cholesterol complex which can effectively deliver the fatty acid and cholesterol to the cells. An example of such a complex is a cyclodextrin/linoleic acid, cholesterol and oleic acid complex. This is advantageous, as it would allow for the replacement of the poorly characterized albumin with a well-defined molecule. The use of cyclodextrin removes the need for the addition of human/animal serum albumin, thereby eliminating any trace undesired materials which the albumin would introduce into the media. The use of cyclodextrin simplifies the addition of specific lipophilic nutrients to a serum-free culture.

Three cyclodextrins which are employable are α-, β-, and γ-cyclodextrins. Among them, β-cyclodextrin appears to be the best. In this invention dealing with the expansion of CD34⁺/CD38⁻ cells, it might be possible to replace the use of human serum albumin with β-cyclodextrin complexed with linoleic acid, cholesterol and oleic acid. However, in other embodiments, any cyclodextrin can be used to include numerous lipophilic substances to the culture.

The lipophilic substances which can be complexed with cyclodextrin include unsaturated fatty acids such as linoleic acid, cholesterol and oleic acid. The linoleic acid, cholesterol and oleic acid are present in effective amounts and can be present in equal proportions such that the total amount is 0.001 to 100 μg/ml, preferably 0.1 to 10 μG/ml. The preparation of such complexes is known in the art and is described, for example, in U.S. Pat. No. 4,533,637 of Yamane et al, the entire contents of which is hereby incorporated by reference.

Iron Source

A source of iron in an effective amount and in a form that can be utilized by the cells is preferably added to the media. The iron can be supplied by transfenin in an effective amount. The transferrin may be derived from animal sera or recombinantly synthesized. It is understood that when transferrin is derived from an animal source, it is purified to remove other animal proteins, and thus is usually at least 99% pure. The transferrin concentration is usually between 80 and 500 μg/ml, preferably between 120 and 500 μg/ml, more preferably between 130 and 500 μg/ml, even more preferably between 275 and 400 μg/ml and most preferably 300 μg/ml. Alternatively, an iron salt, preferably a water soluble iron salt, such as iron chloride (e.g. FeCl₃.6H₂O) dissolved in an aqueous solution such as an organic acid solution (e.g. citric acid) can be used to supply the iron. One mole of iron chloride is usually used for every mole of citric acid. The concentration of iron chloride is 0.0008 to 8 ,g/ml, preferably 0.08 to 0.8 μg/ml, most preferably 0.08 μg/ml.

Insulin Growth Factor

Insulin may also be added to the media of the present invention in an effective amount. The insulin concentration is usually between 0.25 and 2.5 U/ml, more preferably 0.4-2.1 U/ml, most preferably 0.48 U/ml. In the conversion of Units to mass, 27 U=1 mg. Therefore, incorporating the conversion, the insulin concentration is approximately between 9.26 μg/ml and 92.6 μg/ml, more preferably 14.8 μg/ml-77.8 μg/ml, most preferably 17.7 μg/ml. It is again understood that human insulin is more preferable than animal insulin. Highly purified recombinant insulin is most preferred. An insulin-like growth factor such as insulin-like growth factor 1 and insulin-like growth factor 2 may be used in place of insulin in an amount that provides substantially the same result as a corresponding amount of insulin. Thus, the term “insulin growth factor” includes both insulin and insulin-like growth factors.

Additional Components

The addition of other lipids to the above essential reagents could enhance the proliferative potential of precursor cells. These components, however, are preferably not added unless they are necessary for a particular experiment or to grow a particular type of cell. Optionally, triglycerides and/or phospholipids may be included as additional sources of lipid. A preferable source of lipid contains a mixture of neutral triglycerides of predominantly unsaturated fatty acids such as linoleic, oleic, palmitic, linolenic, and stearic acid. Such a preparation may also contain phosphatidylethanolamine and phosphatidylcholine. Another source of lipid is a human plasma fraction precipitated by ethanol and preferably rendered virus-free by pasteurization.

Other ingredients which can optionally be added to the media are cited in the following references: Smith et al, WO 95/06112, Yamane et al, U.S. Pat. No. 4,533,637, Ponting et al, U.S. Pat. No. 5,405,772, Smith et al. U.S. Pat. No. 5,846,529. The entire contents of each of these references are incorporated by reference.

Undesired Components

When the media is to be used to grow cells for introduction into a human patient, the media preferably does not contain ingredients such as bovine serum albumin, mammalian serum, and/or any natural proteins of human or mammalian origin (as explained above). It is preferable that recombinant or synthetic proteins, if they are available and of high quality, are used. Most preferably, the amino acid sequences of the recombinant or synthetic proteins are identical to or highly homologous with those of humans. Thus, the most preferable serum-free media formulations herein contain no animal-derived proteins and do not have even a non-detectable presence of animal protein.

In the most ideal system, optional components that are not necessary are preferably not added to the medium. Such optional components are described in the prior art cited above and may be selected from the group consisting of meat extract, peptone, phosphatidylcholine, ethanolamine, anti-oxidants, deoxyribonucleosides, ribonucleosides, soy bean lecithin, corticosteroids, and EX-CYTE (Serologicals Inc., Kankakee, Ill.), myoinositol, monothioglycerol, and bovine or other animal serum albumin. Furthermore, if the media is being used to maintain or enrich the amount of CD34⁺/CD38⁻ cells in a cell population, it is preferable that growth factors which accelerate differentiation of CD34⁺/CD38⁻ cells to CD34⁺/CD38⁺ cells be avoided. For some uses, it may be desirable to avoid the use of G-CSF, GM-CSF, M-CSF, and erythropoietin.

Ex Vivo Cell Culture

Ex vivo culture techniques hold great promise in the treatment of numerous diseases by 1) generation of HSCs and/or committed progenitors; 2) generation of committed progenitor cells to reduce the period of therapy induced pantocytopenia; 3) purging of malignant cells; 4) transduction of specific genes into hematopoietic cells for gene therapy; and 5) a transport medium/mechanism for HSCs. For these utilities, the use of ex vivo culture of HSC for reconstituting the hematopoictic system has been the most encouraging. Studies have focused not only on the expansion of unfractionated bone marrow cells, but also on the expansion of highly purified HSCs, such as CD34⁺ cells, that have been isolated from cord blood, mobilized peripheral blood, or normal bone marrow. The disadvantage to using these highly purified HSC populations is that the cells may be too few in numbers to support sufficient engraftinent after transplantation. However, the ex vivo expansion of these cells may alleviate such difficulties.

Between 10⁴-10⁵/ml of the HSCs were cultured in said serum-free medium with various growth factors including FLT3, SCF, IL-1β, IL-3, IL-6, G-CSF, GM-CSF, TPO and erythoprotein at concentrations ranging from 1 to 100 ng/ml. The cells were cultured in Costar 24 mm transwell porous inserts placed into 24-well plates or 25 cm² tissue culture flask and incubated at 37° C. with 5% CO² and air in a fully humidified atmosphere. Spent medium and cytokines were replaced every seven days with fresh medium and cytokines. The medium can be changed every 1-7 days, preferably every 2-7 days, more preferably every 3-7 days and most preferably every 7 days. The medium is changed often enough to allow the CD34⁺ cells to grow and proliferate. Unnecessary changing of the media is avoided because of extra time and expense and risk of contamination. The cells were counted on days 0, 3, 7, 10, and 14 of incubation with a hemocytometer and assessed for viability using the trypan blue dye exclusion assays. On days 3, 7, 10 and 14, aliquots of the cells were analyzed for their cell cycle status, immunophenotype, colony forming ability, and number of long-term initiating cells.

It has been observed that conversion of a CD34⁺/CD38⁻ phenotype to CD34⁺/CD38⁺ phenotype is associated with a shift from an uncommitted to a committed phenotype for the HSCs. Thus, assay of the presence or absence of the CD38 marker of CD34⁺ cells allows for rapid determination of the long-term engraftment capability of CD34⁺ cells. It is preferred to harvest the cells when the CD34⁺ population is expanding ex vivo more rapidly or at the same level as the CD34⁺ population is decreasing due to differentiation or death. Thus, the cells should be harvested at or before the total number of CD34⁺ cells begins to decrease. Optimum results are obtained when the cells are cultured for more than 2 days, and possibly up to 8 weeks or longer. The cells are cultured usually from 2 to 14 days, preferably 2 to 7 days, more preferably 2 to 5 days, and most preferably 3 days. It has been discovered that cells, particularly cord blood cells, can be cultured for as long as 8 weeks, for example 4-8 weeks, specifically about 6 weeks, and an increase in the total number of CD34⁺/CD38⁻ cells can be obtained. However, these cells have not been tested for their long-term engrafting ability.

Immunophenotype Analysis

Early progenitors (CD34, CD38, HLA-DR), myeloid markers (CD33, CD14, CD45), lymphocyte markers (CD3, CD7, CDI9), red blood cell markers (glycophorin A) and megakaryocyte/platelet determinants (CD41a) were analyzed using standard staining methods well known in the art and a FACscan three color flow cytometer (Becton-Dickinson, Hiedleberg, Germany).

Transplantation Protocols

Patients who are eligible for allogeneic stem cell replacement therapy may receive a therapeutic amount of the HSC suspension to relieve their corresponding disease state. A skilled artisan knowledgeable in HSC transplantation could determine the optimal transplantation protocol necessary to correct the patient's disease state. An effective amount, typically 1-2×10⁶ cells/kg of body weight of a CD34⁺ HSC population, would be administered to the patient, usually by an intravenous route. Of that CD34⁺ cell population, typically 1-5% of those cells would be CD34⁺/CD38⁻. Not only are HSC transplants useful to replenish bone marrow cells, they are also important in clinical regimens to combat cancer, myeloproliferative diseases and autoimmune diseases via gene therapy. In gene therapy, HSCs may be transfected with a gene of interest and then engrafted into the patient. Cord blood HSCs are excellent candidates for gene transfer because they are immunologically naive and as such will not elicit an immune response when transplanted non-autologously. Furthermore, they provide a specific target into which the gene of interest is transfected outside the patient. The use of HSCs this way avoids many of the targeting problems inherent to gene therapy. Some specific diseases that may be treated by gene therapy include the thalassemias, sickle cell anemia, Fanconi anemia, SCID, chronic granulomarous disease, leukocyte deficiency, Gaucher's disease and several others. HSCs are ideal carriers of these therapeutic genes because they are non-immunogenic and self-perpetuating. Gene therapy protocols are well described in the literature and a person of ordinary skill in the art would be able to determine the best mode for this process. Briefly, the HSCs would be removed from the patient and then transduced with the therapeutic gene (Kohn, 1998; Kohn; 1995; Lu, 1993).

Many methods exist to transfer the therapeutic gene into the cell. A common method is the use of a recombinant retroviral vector containing the therapeutic gene. The retrovirus will infect the cell and integrate into the cell genome. Proper integration will result in the gene being expressed in the cell. The transformed HSCs may then be reintroduced into the patient, thus serving as a mode to transfer the gene into the patient. Many methods exist to determine whether the gene therapy was successful, including correction of the disease state, assay for expression of the therapeutic gene product, presence of a selectable marker and others.

In accordance with the present invention, cells can be transplanted into mammals including animals such as sheep or humans.

Transport of Hematopoietic Stem Cells

Typically, following extraction from the HSC donor, the stem cells must be frozen to stop differentiation and allow proper timing for the transplantation. Unfortunately, many cells die from the freeze/thaw process. Therefore a need exists to keep the HSCs in culture without the freezing-down step. A longer culture time not only provides the patient with a larger window in which the transplantation can take place, but also will allow for the transport of the cells from, for example, clinic to clinic, without the cyropreservation step. The cells can be transported in the media described in this application at a temperature between 4 and 40° C., preferably 20 to 38° C., more preferably 35 to 37° C., most preferably about 37° C. The temperature should be one that prevents a decrease in the total number of CD34⁺/38⁻ cells.

Utility

In the art of tissue culture it has for some time been desired that a serum-free culture system be developed that supports the proliferation of CD34⁺/CD38⁻ cells. This is due to the fact that many therapeutic regimes are being developed which depend upon HSC transplants. Such transplants are useful in the therapy of radiation exposure, immunodeficiency and tumors of the hematopoietic system (leukemias). The serum-free culture system of the present invention can be used to cultivate mixed cell populations which contain CD34⁺ cells to selectively enrich (increase the proportion of) CD34⁺/CD38⁻ cells in the population.

Recent studies have shown that early progenitor/stem (CD34⁺/CD38⁻) cells can be highly purified and can differentiate into all the different hematopoietic lineages in the presence of specific cytokines. These cells have been successfully used in the clinic for transplantation and also have promise for use in gene therapy. However, early CD34⁺/CD38⁻ cells will differentiate into CD34⁺/CD38⁺ cells after 3 days of ex vivo culture, which does not allow for the proper expansion of cells to occur for adult transplantation (Browxneyer 1992).

The serum-free culture system of the present invention, a formulation suitable for use in human therapeutic protocols, has two types of utility in human HSCs transplant therapies. First, the culture system can be used in the expansion of the CD34⁺/CD38⁻ cells that are responsible for repopulating the host HSC population. The culture system of the present invention can be used in the expansion of these early progenitor stem cells that can then be mixed with fresh unfractionated bone marrow and transplanted or transplanted alone. The rationale for this use is that the in vitro treatment allows for differentiation of the early progenitor cells to mature cells, capable of protecting the host from opportunistic diseases that occur during bone marrow transplantations.

The second utility is in “ex vivo purging” protocols. In a therapy of this type, “normal” (non-tumorigenic) CD34⁺ cells that are tainted with tumor cells, either of bone marrow or metastatic origin, are placed into in vitro culture in the medium of the present invention. The mixture of normal bone marrow cells and tumor cells is then treated with reagents that are preferentially cytotoxic for the tumor cells. Alternatively, the tumor cells can be selectively depleted from the culture using immobilized antibodies that specifically bind to the tumor cells. The “purged” bone marrow is then transplanted back into the patient. The culture system of the present invention is suitable for storing the cells when they are removed from the human body and is also particularly useful for growing the cells when they are removed from the human body for at least 3 days. The medium is especially adapted to selectively promote the growth of CD34⁺/CD38⁻ cells so that a mixed culture of cells can be enriched in CD34⁺/CD38⁻ cells and the CD34⁺/CD38⁻ cells can be returned to a patient in need of the cells. The culture system is also useful for growing CD34⁺/CD38⁻ cells after they have been separated from other cells. After the CD34⁺/CD38⁻ cells have been grown to increase the number of cells, they can be given to a human patient for known therapies.

A third utility is a “selection” process, which is a necessary step for ex vivo expansion. Ex vivo expansion, in addition to rapid and reliable recovery from dose-intensive therapy, would permit either smaller quantities of bone marrow or peripheral blood progenitor cells. Preliminary clinical investigations have shown the potential utility of ex vivo expansion. In these studies, hematopoietic progenitor cells are isolated and expanded in bioreactors containing media with hematopoietic growth factors. Usually fresh hematopoietic progenitor cells are used, but one group reported that CD34-selected cells could be cryopreserved, thawed, and then expanded. If small aliquots of HSCs could be expanded and used to accelerate hematopoietic recovery after dose-intensive therapy, then the problems with stem cell harvesting, both from marrow and peripheral blood would be markedly diminished. Problems with general anesthesia and obtaining large volumes of marrow would be eliminated; problems with venous access and long periods of apheresis over several days would be eliminated.

The invention is illustrated by the Examples below, which are not intended to be limiting of the scope of the invention.

### EXPERIMENTAL EXAMPLES

In the present studies, the ability of the serum-free media QBSF-60 to maintain or support the ex vivo expansion and/or maintenance of HSCs was analyzed. Adult human bone marrow CD34⁺ cells were cultured in QBSF-60 with or without FBS, in the presence of 5 ng/ml IL-3, 5 ng/ml IL-6 and 10 ng/ml SCF, and were analyzed at days 3, 7 and 14 of culture for expansion, phenotype, clonogenic ability and cycling status. The human-sheep xenograft model of human hematopoiesis was utilized to correlate the engraftment potential of the expanded cells at each time point of culture with the results obtained in the in vitro assays.

Material and Methods

Human Donor Cell Preparation.

Heparinized human bone marrow (HBM) was obtained from healthy donors after informed consent. Adult sheep bone marrow (SBM) was obtained from the posterior iliac crest of normal adult sheep following standard procedures that had been approved by the University of Nevada Institutional Animal Care and Use Committee (IACUC).

Low-density bone marrow mononuclear cells (BMNC) were separated by a Ficoll density gradient (1.077 g/ml) (Sigma, St. Louis, Mo.) and washed twice in Iscove's modified Dulbecco's media (IMDM) (Gibco Laboratories, Grand Island, N.Y.). BMNC from each donor were enriched for CD34⁺ cells using magnetic cell sorting (Miltenyi Biotec Inc., Auburn, Calif.).

Ex vivo Expansion of CD34⁺ Cells.

10⁵ CD34⁺ cells/ml were cultured for 3, 7 or 14 days in QBSF-60 (Quality Biological, Gaithersburg, Md.) with or without FBS (Hyclone, Logan, Utah) in the presence of the following cytokines: IL-3 (5 ng/ml), IL-6 (5 ng/ml), and SCF (10 ng/ml) (Peprotech, Rocky Hill, NJ).

Clonogenic Assays.

Assays for clonogenic progenitors were performed in triplicate in MethoCult GF H4434 (StemCell Technologies Inc., Vancouver, Canada) on CD34⁺ cells that were freshly purified or expanded for 3, 7 and 14 days of culture. Cultures were incubated in a humidified incubator at 37° C. in 5% CO₂ air. After 14 days, colonies were counted and categorized according to standard criteria.

Proliferation and Phenotypic Analysis.

The ex vivo expansion of the purified CD34⁺ population was determined at each time point by counting the content of hematopoietic cells in each culture flask. The hematopoietic cells were then further analyzed for stem cell and lineage content by flow cytometry using monoclonal antibodies against CD3, CD11b, CD15, CD33, CD34 and CD38 (Becton Dickinson Immunoctyometry Systems [BDIS], San Jose, Calif.).

Creation of Human Sheep Chimeras.

Human HSCs were transplanted into thirty-six fetal sheep (19 primary recipients, 17 secondary recipients) at 55-60 days of gestation utilizing the following transplantation procedure. In short, freshly isolated or cultured 9×10⁵ CD34⁺ cells were injected intraperitoneally in a 0.5 ml volume into 55-60 day-old fetal sheep. The transplanted sheep were analyzed for donor (human) cell engraftment in bone marrow, thymus, liver, spleen and peripheral blood at 9 weeks after transplantation (118-123 days of gestation) and after birth at 1 week, 3 and 8 months of age. Of the 19 transplanted primary recipients, 17 were available for analysis (8 were sacrificed at 60 days post-transplant, and 9 were born alive). Secondary transplants were performed using 6×10⁶ BMNC from the primary recipients. Of 17 secondary recipients, all of the transplanted sheep were available for analysis. Seven were sacrificed at 60 days post-transplant and the other 7 were born live.

Assessment of Human Donor Cell Engraftment.

The presence of donor cells in hematopoietic tissues of the recipients (blood, marrow, liver, spleen, and thymus) was determined at intervals, post-transplantation, using flow cytometric analysis and hematopoietic progenitor assays. Flow cytometric analysis of the cell populations was performed on a FACScan (BDIS). Monoclonal antibodies to various cluster designations (CDs) directly conjugated with FITC or PE were used according to the manufacturer's recommendation. The cluster designations included: CD45, CD14, CD34, CD20, CD33, CD3, CD7, CD56, CDIO, CD4, CD8 (BDIS) and glycophorin A (Immunotech, Miami, Fla.).

Statistical Analysis.

Results are expressed as mean±standard error of the mean (SEM). Comparisons between experimental results were determined by two-sided, non-paired Student's test analysis. A p value <0.05 was considered statistically significant.

### Example 1

Evaluation of ex vivo Expansion of CD34⁺ Cells in QBSF-60 Serum Free Media.

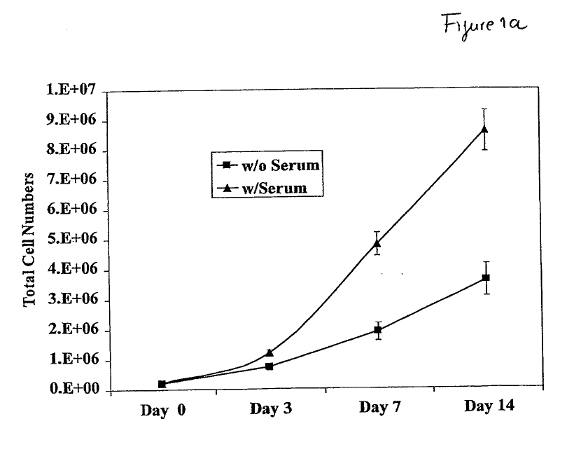

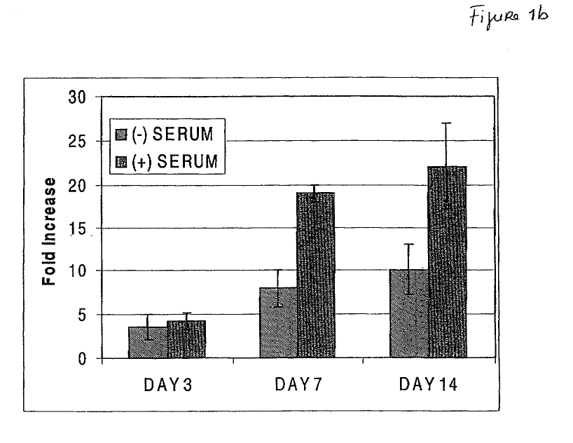

Ex vivo expansion of human bone marrow CD34⁺ in serum free media, QBSF-60, supplemented with low levels of IL-3 (5 ng/ml), IL-6 (5 ng/ml) and SCF (10 ng/ml) was evaluated in comparison to serum-supplemented cultures for 3, 7 and 14 days. FIG. 1A shows the total cell numbers found at day 0 to day 14 that were cultured with or without serum, while FIG. 1B shows the fold increases in the total numbers of cells obtained at day 3, day 7 and day 14 of culture. At day 7, the proliferation rate of the cells grown in serum is 3 times that of the cells grown in serum-free conditions, and roughly 2.5 times at day 14. These results show that cells cultured in the presence of serum proliferated at least 2 times more rapidly than cells without serum.

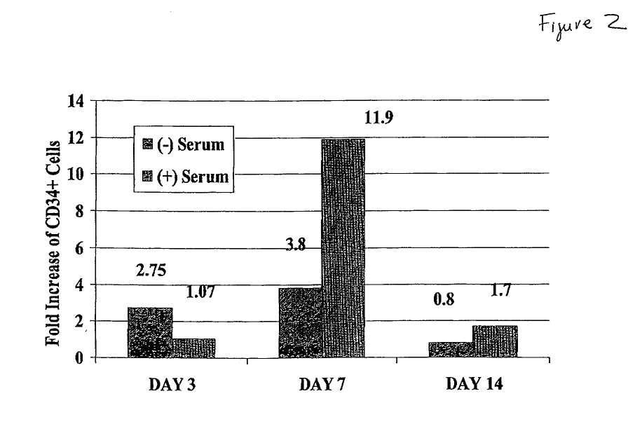

Although the absolute cell numbers are increasing as shown in FIG. 1B, the number of CD34⁺ cells have an initial increase but then decrease in number due to cell differentiation. Those cells that differentiate lose their CD34⁺ marker and are no longer detectable by the CD34⁺ monoclonal antibody. As shown in FIG. 2, the maximal fold increase of CD34⁺ cells over day 0 was obtained after 7 days of culture with serum, with a 3.8 fold increase in CD34⁺ cells over day 0 when grown in serum-free medium.

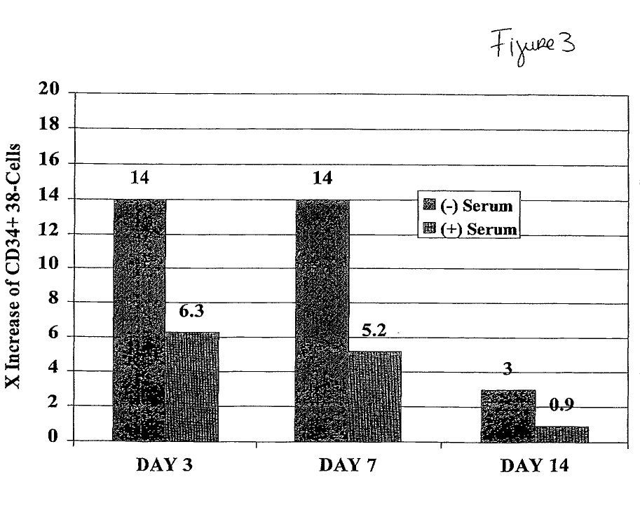

However, when the more primitive CD34⁺/CD38⁻ phenotype was analyzed at the same time points of culture, this population of cells showed a significant expansion at day 3 and 7 in the fraction of cells cultured without serum (FIG. 3). CD34⁺/CD38⁻ cells grown in serum-free medium expanded 14 fold over day 0 at both day 3 and day 7 time points.

Evaluation of the in vivo Engraftment Capability of the Expanded Cells.

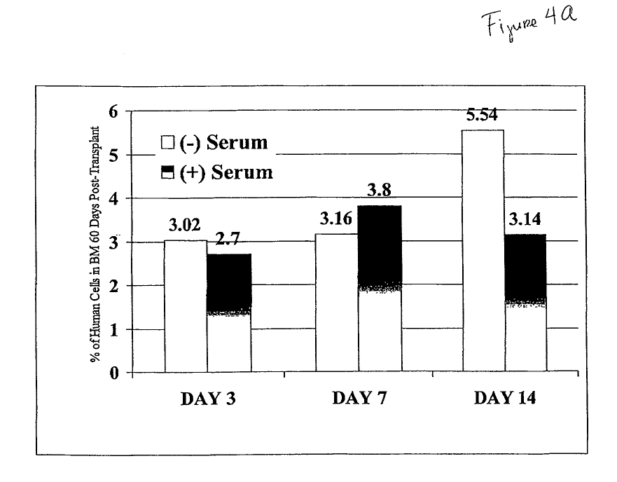

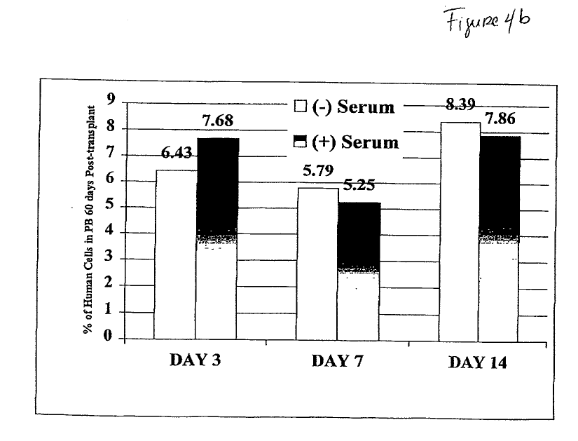

The human/sheep xenograft model was utilized to determine how culture conditions and the number of days in culture affected the in vivo engrafting capability of the CD34⁺ enriched ex vivo expanded cells when compared to freshly isolated cells. Approximately 9×10⁵ freshly isolated (n=5) or expanded (n=14) human BM CD34⁺ cells were transplanted into 19 primary sheep recipients. The cells were expanded either 3, 7, or 14 days in either serum-containing or serum-free medium. The primary recipients were subsequently evaluated at 60 days post-transplant and after birth at 1 week, 3 and 8 months of age. FIG. 4A shows the percentage of human cells persisting in the BM of the primary recipient sheep 60 days post-transplant, while FIG. 4B shows the percentage of human cells persisting in the peripheral blood of the primary recipient sheep 60 days post-transplant. From the data, it is apparent that the expanded cell populations gave rise to multilineage engraftment and differentiation in the bone marrow and peripheral blood of these primary recipients as examined 60 days post-transplant. However, the highest levels of donor cell engraftment seen in both peripheral blood and marrow were achieved with the cells that were cultured under serum-free conditions for 14 days.

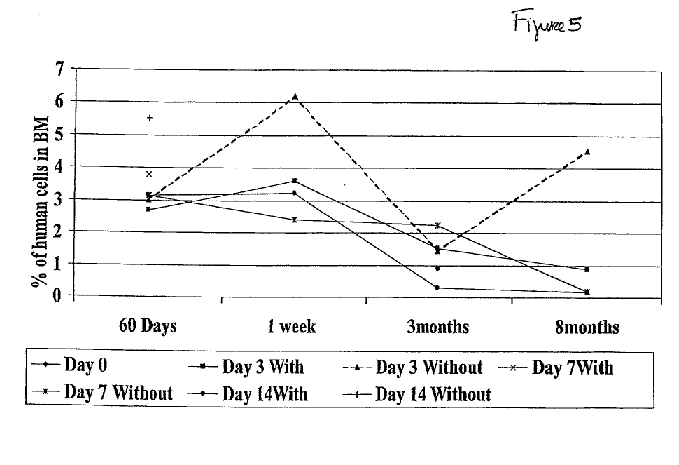

The percentage of human cells in the bone marrow under various culture conditions is shown in FIG. 5. The long-term engraftment of human cells in the BM of sheep was analyzed at 60 days, 1 week, 3 months, and 8 months (335 days) post-transplant. At 8 months of age (335 days post-transplant) the levels of human cells that were cultured for 3 days under serum-free conditions in the bone marrow of sheep was 4.58%, while sheep transplanted with human HSCs cultured for 3 days with serum and 7 or 14 days both with and without serum exhibited less than 1% donor cells.

Ability of Cultured Cells to Engraft Secondary Recipients.

Subsequently, the ability of human cells that were present within these primary recipients to engraft secondary fetal sheep recipients was evaluated. It has been previously demonstrated that populations of highly primitive human stem/progenitor cells readily engraft within secondary recipients while the more differentiated progenitors do not, thus enabling direct evaluation of whether differentiation has occurred during the ex vivo culturing process.

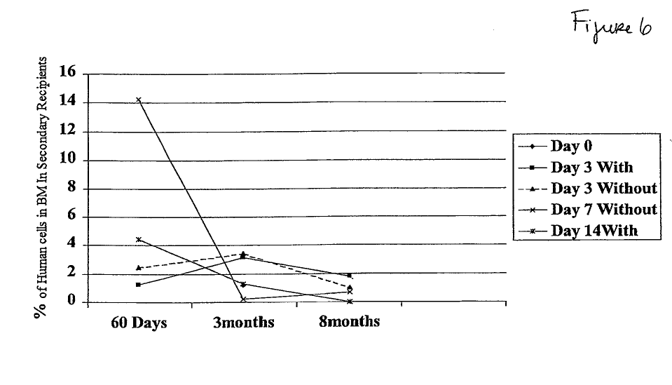

To this end, bone marrow aspirates were obtained from the primary sheep at 60 days post-transplant, and 6×10⁶ of the resultant mononuclear cells were transplanted into each fetal sheep recipient at 55 days of gestation (n=17). FIG. 6 shows the short-term engraftment levels within the marrow of the secondary recipients. The highest levels (>14%) of human cells present were those that were expanded for 7 days in the absence of serum. However by 3 months, these levels decreased to the lowest of any of the groups and remained low throughout the remainder of the study. At 3 and 8 months post-transplant, the secondary recipient sheep that received cells that were expanded for 3 days under serum-free conditions exhibited the highest levels of marrow engraftment (around 2%). Sheep that received secondary transplants with cells that had been cultured for 14 days exhibited engraftment at the early time points (>4%). However, all evidence of engraftment was gone by 8 months post-transplant, demonstrating that the long-term repopulating cells were lost during this lengthy culture period.

Discussion

In human/sheep chimeric animals, human HSC 1) colonizes the bone marrow, 2) persists for long periods, 3) is capable of multilineage differentiating in response to human-specific hematopoietic regulatory cytokines, 4) retains its ability to respond to human cytokines, and 5) retains it ability to engraft/differentiate in secondary recipients.

The ability of human cells isolated from bone marrow of primary human/sheep chimeric animals has been used to engraft the bone marrow of secondary preimmune fetal sheep recipients to establish the relative specificity of this model. This has enabled us for the first time to evaluate the in vivo engraftment/proliferation/differentiation potential of different human HSC populations. A major focus in experimental hematology is the delineation of conditions that would allow HSC to be manipulated in vitro in such a way that they could expand in number yet maintain all of the characteristics that define an HSC. The definition of such strategies would impact profoundly on both clinical HSC transplantation and gene therapy. Numerous studies have demonstrated that the absolute number of cells that carry surface markers are indicative of HSC can indeed be increased ex vivo. It has now been shown that these cells often sacrifice their ability to provide reliable engraftment in order to increase in number ex vivo in response to various cytokines, notably IL-3 and SCF. Since to date, no ex vivo assay system has been developed that can accurately predict the engraftment potential of HSC, it is imperative that studies evaluating the expansion of putative HSC populations ex vivo be preformed. These studies include those in which the ability of the expanded cells to engraft both primary and secondary recipients is examined.

Thus far, the majority of studies that have demonstrated expansion of long-term engrafting HSC have accomplished this by employing culture systems that combine cytokine stimulation with support of a feeder cell layer. While this system does in fact allow HSC expansion, it can be argued that the in vitro incubation of an HSC graft with a feeder layer with ill-defined pathogenic potential is unlikely to find clinical application. For this reason, we set out in the present studies to develop a straightforward, serum-free liquid culture system that is both reproducible and would be readily applicable to clinical HSC transplantation. A population of adult bone marrow cells that were highly enriched for the surface marker CD34 were employed. It could be argued that this population of cells is very heterogeneous and does contain cells that have already committed to various lineages. Additionally, previous studies have provided evidence that expansion may be greater if cells with a more primitive phenotype are employed. However, we reasoned that since the majority of transplants are currently performed using CD34-enriched cells, the derivation of methods for expanding the number of long-term engrafting cells within this population would be of more direct clinical utility.

In the present studies, QBSF-60 and low concentrations of IL-3, IL-6, and SCF were used, both in the presence and absence of serum, to investigate whether a population of CD34-enriched cells from adult bone marrow could be expanded and still maintain their ability to engraft both primary and secondary recipients using the human/sheep xenograft model of human hematopoiesis. CD34⁺ cells were cultured for 14 days and analyzed at 3, 7 and 14 days for expansion, phenotype, and in vivo engraftment potential. Although both the cells cultured with serum and in its absence exhibited a progressive expansion in total cell number, the group cultured with serum exhibited more than twice the expansion seen in the group without serum at all time points. However, both groups showed a decrease in the number of CD34⁺ cells throughout the 14-day culture period. More importantly, however, the population of CD34⁺/CD38⁻ cells persisted in significantly higher numbers in the group cultured without serum, producing maximal output of CD34⁺/CD38⁻ cells at days 3 and 7. In addition, a higher total clonogenic potential in the serum-free cultures was observed. Thus, from the ex vivo data, it can be concluded that serum-free conditions provide better support form the more primitive cells.

In order to evaluate the in vivo engraftment potential of the expanded hematopoietic cells, fetal sheep recipients were transplanted with an identical number of either fresh or cultured cells. The highest level of long-term engraftment was obtained with the fraction of cells cultured for 3 days in the absence of serum. These results in the primary recipient suggest that expanding the number of primitive HSC during at least 3 days of culture has been successful, since transplanting the same number of expanded cells yielded a higher level of engraftment.

In order to further distinguish between primitive progenitors that could provide fairly durable engraftment in primary recipients and truly long-term repopulating HSC, marrow mononuclear cells from the primary recipients were used to transplant secondary fetal sheep recipients. The secondary recipients were far more informative than the primary sheep with regard to assessing functional differences between the cells from different culture conditions. While cells from sheep that had been made chimeric with HSCs expanded for 3, 7 and 14 days in the absence of serum were all capable of engrafting secondary recipients, the cells cultured for 14 days were exhausted by 8 months post-transplant, demonstrating that long-term repopulating HSC were not maintained throughout the 14-day culture period. By contrast, HSC cultured for 3 or 7 days without serum were both capable of providing long-term engraftment within the secondary recipients. However, the level of engraftment seen with the cells cultured for 3 days was far more substantial, corroborating the ex vivo results.

In conclusion, QBSF-60 could be used for ex vivo HSC expansion and potentially HSC gene therapy, since it is able to expand human HSC for up to 7 days in culture while maintaining both a primitive phenotype and the ability of the cells to engraft in physiologically relevant human-to-sheep xenograft model of human hematopoiesis.

### Example 2

A preparation of 32 mls of fresh cord blood was prepared and subsequently analyzed by flow cytometry to be 39.5% pure. Thus about 6×10⁵ CD34⁺ cells were present and isolated from the fraction. In these studies the volume of cells used were adjusted to represent the numbers of CD34⁺ cells/culture/fetus. The experimental groups were divided into three sections: A, B and C. Group A consisted of preimmune fetal sheep (58 days old; term: 145 days). Each fetus in this group received an injection of 4×10⁴ starting CB CD34⁺ cells intraperitoneally. Group B consisted of 4 preimmune fetal sheep (56-59 days old). Each fetus in this group received 4×10⁴ equivalent CB CD34⁺ cells that had been cultured for 4 days at 37° C. in QBSF60 with 5ng/ml SCF, 10 ng/ml Flt3 and 10 ng/ml TPO. The culture was set up using 1.6×10⁵ CB CD34⁺ cells in 4 ml QBSF60 containing the above cytokines. At the end of 4 days of culture, the content was divided into 4 equal aliquots, each of which was used to transplant one fetus. No cell count or flow studies were done. Group C consisted of 4 preimmune fetal sheep (54-62 days old). Each fetus in this group received 4×10⁴ equivalent CB CD34⁺ cells that had been cultured for 4 days at 37° C. in QBSF60 with 50 ng/ml SCF, 100 ng/ml Flt3, and 100 ng/ml TPO. The culture was set up with 1.6×10⁵ CB CD34⁺ cells in 4 ml QBSF60 with the above cytokines. At the end of 4 days of culture, the content was divided into 4 equal aliquots, each of which was used to transplant one fetus. No cell counts or flow studies were done.

The primary recipients were sacrificed on day 60 post-transplant (i.e., about 1 month before birth). All eight long bones from each recipient were flushed thoroughly using IMDM/10% FCS and the cells were pooled. The presence of human cells in the pooled BM cells from each animal was evaluated by flow cytometry (results shown in Table 2). Furthermore, the human hematopoietic progenitor content was obtained using culture in methylcellulose and karyotyping (as described in a number of our previous publications) (results shown in Table 3). As shown in Tables 2 and 3, each group (A, B, and C) contain HSCs in the BM of the primary recipients at 60 days post-transplants. The remaining cells from each animal were then pooled and subjected to panning (as described earlier) for the isolation of human CD45+ cells to be used for transplant into secondary fetal sheep recipients (see below).

TABLE 2
Donor (human) cell activity in BM of primary
recipients at 60 days post-transplants.

% Human Cell Activity*

| Group | CD45 | CD34 | CD3 | GlyA |
|---|---|---|---|---|
| A | 3.9 + 0.2 | 0.28 + 0.06 | 2.4 + 0.7 | 8.9 + 3.2 |
| B | 4.1 + 0.9 | 0.21 + 0.05 | 3.9 + 1.2 | 7.1 + 1.6 |
| C | 3.8 + 0.4 | 0.31 + 0.11 | 1.3 + 0.6 | 5.7 + 1.3 |

*Each value represents mean ± 1 SEM of results obtained from either 4 (groups A and B) or 3 (group C) animals. The remaining fetus in group C died of infection on day 6 post-transplant and was lost to the study.

TABLE 3
Donor (human) hematopoietic progenitor activity in BM
of primary recipients at 60 days post-transplant.*

| Group | CFU-Mix | CFU-GM |
|---|---|---|
| A | 5.8 + 1.2 | 9.8 + 2.0 |
| B | 8.6 + 3.1 | 14.7 + 3.3 |
| C | 6.2 + 1.7 | 10.8 + 2.1 |

*Each value represents mean ± 1 SEM of results obtained from either 4 (groups A and B) or 3 (group C) recipients.

Human CD45+ cells were isolated from bone marrow of primary recipients (pooled for each group) by panning as described. The total numbers of CD45+ cells obtained from each group are shown in Table 4.

TABLE 4
Total numbers of human CD45+ cells obtained from BM of
each group of primary recipients at 60 days post-transplant.

| | |
|---|---|
| Group A | 1.3 × 10⁶ cells |
| Group B | 1.7 × 10⁶ cells |
| Group C | 1.6 × 10⁶ cells |

The CD45+ cells were subsequently transplanted into secondary animals as shown below. There were three experimental groups: D, E, and F. Group D consisted of 4 preimmune fetuses (55-59 days old). Each fetus in this group was injected with 2.5×10⁵ CD45+ cells from primary group A. Group E consisted of 4 preimmune fetuses (55-59 days old). Each fetus in this group was injected with 2.15×10⁵ CD45+ cells from primary group B. Group F consisted of 4 preimmune fetuses (55-59 days old). Each fetus in this group was injected with 2.1×10⁵ CD45+ cells from primary group C.

All animals in these groups were sacrificed on day 60 post-transplant (i.e. about 1 month before birth). Bone marrow cells were obtained from all 8 long bones from each animal using IMDM/10% FCS and pooled. Pooled cells from each secondary recipient were evaluated for human origin via flow cytometry (results shown in Table 5). The presence of human hematopoietic progenitor cells was analyzed by methylcellulose, and karyotyping (results shown in Table 6). Tables 5 and 6 show that each group of secondary transplant recipients again has the presence of HSCs in their BM. The results demonstrate the long-term engrafting ability of the transplanted HSCs. The remaining cells from each animal in the group were pooled and used for transplant into tertiary recipients (see below).

TABLE 5
Donor (human) cell activity in BM of secondary
recipients at 60 days post-transplant.

% Human Cell Activity*

| Group | CD45 | CD34 | CD3 | GlyA |
|---|---|---|---|---|
| D | 5.3 + 0.2 | 0.15 + 0.06 | 0.9 + 0.3 | 4.8 + 0.7 |
| E | 8.1 + 2.5 | 0.09 + 0.04 | 1.5 + 0.3 | 5.0 + 1.0 |
| F | 6.2 + 1.1 | 1.9 + 0.04 | 1.6 + 0.09 | 3.9 + 0.3 |

*Each value represents mean ± 1 SEM of results from 4 animals/group.

TABLE 6
Donor (human) hematopoietic progenitor activity in bone
marrow of secondary recipients at 60 days post-transplant.*

| Group | CFU-Mix | CFU-GM |
|---|---|---|
| D | 5.3 + 1.2 | 11.4 + 3.9 |
| E | 5.0 + 1.4 | 7.2 + 2.2 |
| F | 4.2 + 1.7 | 9.1 + 3.1 |

*Each value represents mean ± 1 SEM of results from 4 animals/group.

Human CD45+ cells were isolated from pooled BM of each group by panning as described. The numbers of CD45+ cells obtained from each group are shown in Table 7.

TABLE 7
Total CD45+ cells isolated from BM of secondary recipients groups

| | |
|---|---|
| Group D | 2.3 × 10⁶ cells |
| Group E | 1.3 × 10⁶ cells |
| Group F | 1.9 × 10⁶ cells |

The cells were transplanted into tertiary recipients as indicated below. The experimental groups were of three: G, H, and I. Group G consisted of 3 preimmune fetuses (58 days old). Each fetus in this group was injected with 7×10⁵ CD45+ cells from secondary group D. Group H consisted of 3 preimmune fetuses (58 days old). Each fetus in this group was injected with 4×10⁵ CD45+ cells from secondary group E. Group I consisted of 3 preimmune fetuses (58 days old). Each fetus in this group was injected with 4.5×10⁵ CD45+ cells from secondary group F.

These animals were sacrificed at 40 days post-transplant and their BM cells were analyzed for human origin by flow cytometry (results shown in Table 8). Table 8 shows that all groups of the tertiary transplant recipients have the presence of HSCs in their BM. These results demonstrate long-term engrafting ability of the transplanted HSCs. No progenitor assays were done.

TABLE 8
Donor (human) cell activity in BM of tertiary
recipients at 40 days post-transplant.

| | % Human Cell* | | |
|---|---|---|---|
| Group | CD45 | HLA-DR | GlyA |
| G | 2.1 + 0.5 | 5.3 + 1.2 | 3.3 + 1.0 |
| H | 1.9 + 0.4 | 3.2 + 0.7 | 3.9 + 3.9 |
| I | 1.9 + 0.8 | 4.1 + 1.7 | 9.3 + 2.9 |

*Each value represents mean ± 1 SEM of results from 3 animals/group.

### Example 3

The studies in Example 4 were designed to determine whether culturing CB cells in QBSF60 with various growth factors for 7 or 14 days affects their in vivo engraftment ability. A total of 7.3×10⁵ CD34⁺ cells (92.1% purity) were isolated from a fresh CB (28.6 ml). The cells were used in culture and transplants as indicated below.

There were three experimental groups: J, K, and L. Group J consisted of 3 preimmune fetuses (54-57 days old). Each fetus in this group was injected with 4×10⁴ equivalent CB CD34⁺ uncultured cells. Group K consisted of 4 preimmune fetuses (58-62 days-old). Each fetus in this group was injected with 4×10⁴ equivalent CB CD34⁺ cells which were cultured for 7 days in QBSF60 with 50 ng/ml SCF, 100 ng/ml Flt3, and 100 ng/ml TPO. Group L consisted of 4 preimmune fetuses (55-59 days old). Each fetus in this group was injected with 4×10⁴ equivalent CB CD34⁺ cells cultured for 14 days in QBSF60 with 50 ng/ml SCF, 100 ng/ml Flt3, and 100 ng/ml TPO.

At the end of 7 days and again 14 days (½ volume was removed on day 7 and replaced with fresh medium with cytokines) cell numbers and CD34⁺ cells were determined, and the cells transplanted accordingly. At day 7 there was a 7.2 fold increase in CD34⁺ cells and 19.3 fold increase in cell numbers.

At day 14 there was an 11.6 fold increase in CD34⁺ cells and a 32.8 fold increase in total cell number. Transplantations were based on injecting 4×10⁴ CD34⁺ cells into each fetus. The primary recipients were sacrificed on day 60 post-transplant and BM cells were obtained from all 8 long bones and pooled for each group. The pooled cells were analyzed for human origin and progenitor activity (results shown in Table 9). As shown in Table 9, HSC activity is found in groups J and K, but is exceptionally low in group L.

TABLE 9
Donor (human) cell/progenitor activities in BM of primary
recipients at 60 days post-transplant.

| | % Human cell/progenitor* | | | | |
|---|---|---|---|---|---|
| Group | CD45 | CD34 | GlyA | CFU-Mix | CFU-GM |
| J | 3.4 + 0.3 | 0.15 + 0.03 | 6.6 + 0.4 | 5.2 + 0.5 | 7.9 + 1.6 |
| K | 1.2 + 0.4 | 0.07 + 0.03 | 2.1 + 0.4 | 2.1 + 0.2 | 4.1 + 0.6 |
| L | <0.03 | 0 | <0.05 | 0 | 0 |

*Each value represents mean ± 1 SEM of results from either 3 (group J) or 4 animals (groups K and L) per group.

The secondary transplants were done in three experimental groups: M, N, and O. Group M consisted of 3 preimmune fetuses (60 days old). Each fetus in this group was injected with 9×10⁶ BM cells from primary recipient group J. Group N consisted of 3 preimmune fetuses (60 days old). Each fetus in this group was injected with 9×10⁶ BM cells from primary recipient group K. Group O consisted of 4 preimmune fetuses (57-62 days old). Each fetus in this group was injected with 9×10⁶ BM cells from primary recipient group L. Because bone marrow from group L (i.e. animals transplanted with 14 days cultured cells) did not contain significant numbers of human CD45+ cells, whole unsupported BM mononuclear cells were used for secondary transplants rather than isolated CD45+ cells. All recipients were sacrificed on day 60 post-transplant. Bone marrow cells from all long bones were obtained and evaluated as previously described (results shown in Table 10). Table 10 shows that HSCs that were cultured for 7 days are present, but no activity of HSCs that were cultured for 14 days is found in the secondary recipients. The results indicate that HSCs grown for 14 days under these conditions may not support long-term engraftment.

TABLE 10
Donor (human) cell/progenitor activities in BM of secondary
recipients at 60 days post-transplant.

| | % Human cell/progenitor* | | | | |
|---|---|---|---|---|---|
| Group | CD45 | CD34 | GlyA | CFU-Mix | CFU-GM |
| M | 5.2 + 1.0 | 0.21 + 0.05 | 5.8 + 0.7 | 6.2 + 2.0 | 16.8 + 4.9 |
| N | 0.6 + 03 | 0.06 + 0.03 | 1.1 + 0.5 | 1.6 + 0.5 | 3.2 + 1.1 |
| O | 0 | 0 | 0 | 0 | 0 |

*Each value represents mean ± 1 SEM of results from either 3 (groups M and N) or 4 (group C) animals/group.

The invention being thus described, various modifications of the materials and methods set forth will be obvious to one of skill in the art. Such modifications are within the scope of the invention as defined by the claims below.

What is claimed is:

1. A method for determining the suitability of a cell population for transplantation into a patient requiring a hematopoietic stem cell transplant, comprising the steps of:
obtaining a sample of cells including hematopoietic stem cells; and
determining the number of CD34⁺/CD38⁻ cells in said sample.

2. The method of claim 1, wherein said hematopoietic stem cells are derived from human umbilical cord blood or human bone marrow or are obtained by purifying whole blood.

3. The method of claim 1, wherein said hematopoietic stem cells are grown ex vivo under conditions which increase the total number CD34⁺/CD38⁻ cells.

4. The method of claim 1, wherein flow cytometry is used to determine the number of CD34⁺/CD38⁻ cells.

5. A method of gene therapy, comprising the steps of:
culturing a sample of hematopoietic stem cells under conditions which maintain an effective amount of cells having long-term engraftment phenotype;
transferring a therapeutic gene to correct a genetic defect into said hematopoietic stem cells either before or after said culturing;
transplanting a therapeutic amount of hematopoietic stem cells into a patient requiring said gene therapy; and
measuring the amount of CD34⁺/CD38⁻ cells either before, after, or during said transplantation.
